(19) United States
(12) Patent Application Publication
(10) Pub. No.: US 2023/0069040 A1
(43) Pub. Date: Mar. 2, 2023

(54) EAR-WEARABLE ELECTRONIC DEVICE INCLUDING IN-EAR RESPIRATION SENSOR

(71) Applicant: STARKEY LABORATORIES, INC., Eden Prairie, MN (US)

(72) Inventors: Roy Talman, Tel Aviv (IL); Yaniv Ganor, Tel Aviv (IL); Lior Weizman, Tel Aviv (IL); Neta Aviel, Tel Aviv (IL); Nitzan Bornstein, Tenafly, NJ (US)

(21) Appl. No.: 17/894,314

(22) Filed: Aug. 24, 2022

Related U.S. Application Data
(60) Provisional application No. 63/238,950, filed on Aug. 31, 2021.

(51) Int. Cl. A61B 5/08; A61B 5/0205; A61B 5/024

(52) U.S. Cl. CPC A61B 5/0816; A61B 5/0205; A61B 5/02416; A61B 2562/0219

(57) ABSTRACT

An ear-wearable electronic device comprises a motion sensor configured to generate motion information and a first respiration rate estimated using the motion information. A

PPG sensor is configured to generate PPG data and a second respiration rate estimate using the PPG data. A processor is configured to produce a respiration rate estimate using the first and second respiration rate estimates. A communication device is configured to communicate the respiration rate estimate to one or both of an external electronic device and a cloud database.

### RELATED APPLICATIONS

This application claims the benefit of U.S. Provisional Application No. 63/238,950, filed Aug. 31, 2021, the entire content of each of which is hereby incorporated by reference.

### TECHNICAL FIELD

This application relates generally to devices and sensing methods for measuring respiration rate from within an ear, such devices including ear-wearable electronic devices, hearing aids, commercial hearables, earbuds, personal amplification devices, and physiologic/biometric monitoring devices.

### BACKGROUND

Respiration rate (RR), referred to interchangeably as respiratory rate, is an important vital sign to check when assessing the health of a patient. This vital sign provides information on clinical deterioration of a patient, is predictive of cardiac arrest, and supports the diagnosis of severe pneumonia, for example. Respiration rate responds to a variety of stressors, including stress, cognitive load, cold, and hyperthermia, for example. While exercising, respiration rate can serve as a good marker of physical effort and fatigue.

### SUMMARY

Embodiments are directed to a method of determining respiration rate using an ear-wearable electronic device. The method involves obtaining motion information from a motion sensor of the ear-wearable electronic device, and generating a first respiration rate estimate using the motion information. The method also involves obtaining photoplethysmographic (PPG) data from a PPG sensor of the ear-wearable electronic device, and generating a second respiration rate estimate using the PPG data. The method further involves producing a respiration rate estimate using the first and second respiration rate estimates.

Embodiments are directed to an ear-wearable electronic device comprising a motion sensor configured to generate motion information and a first respiration rate estimated using the motion information. A PPG sensor is configured to generate PPG data and a second respiration rate estimate using the PPG data. A processor is configured to produce a respiration rate estimate using the first and second respiration rate estimates. A communication device is configured to communicate the respiration rate estimate to one or both of an external electronic device and a cloud database.

Embodiments are directed to a method of determining respiration rate using an ear-wearable electronic device comprising obtaining motion information indicative of body motion from a motion sensor of the ear-wearable electronic device, generating, using a processor of the device, a respiration rate estimate using the motion information, and communicating the respiration rate estimate to one or both of an external electronic device and a cloud database.

Embodiments are directed to an ear-wearable electronic device comprising a motion sensor configured to generate motion information indicative of body motion, a processor configured to produce a respiration rate estimate using the motion information, and a communication device configured to communicate the respiration rate estimate to one or both of an external electronic device and a cloud database.

Embodiments are directed to a method of determining respiration rate using an ear-wearable electronic device comprising obtaining photoplethysmographic (PPG) data from a PPG sensor of the ear-wearable electronic device, generating, using a processor of the device, a respiration rate estimate using the PPG data, and communicating the respiration rate estimate to one or both of an external electronic device and a cloud database.

Embodiments are directed to an ear-wearable electronic device comprising a photoplethysmographic (PPG) sensor configured to generate PPG data, a processor configured to produce a respiration rate estimate using the PPG data, and a communication device configured to communicate the respiration rate estimate to one or both of an external electronic device and a cloud database.

The above summary is not intended to describe each disclosed embodiment or every implementation of the present disclosure. The figures and the detailed description below more particularly exemplify illustrative embodiments.

### BRIEF DESCRIPTION OF THE DRAWINGS

Throughout the specification reference is made to the appended drawings wherein:

FIG. 1 is a block diagram of a representative ear-wearable electronic device which includes a respiration rate sensor in accordance with any of the embodiments disclosed herein;

FIG. 2 illustrates a respiration sensor integral to an ear-wearable electronic device in accordance with any of the embodiments disclosed herein;

FIG. 3 illustrates a method implemented by the ear-wearable electronic device illustrated in FIG. 2;

FIG. 4 illustrates a respiration sensor deployed in an ear-wearable electronic device in accordance with any of the embodiments disclosed herein;

FIG. 5 illustrates a respiration sensor integral to an ear-wearable electronic device in accordance with any of the embodiments disclosed herein;

FIG. 6 illustrates a method implemented by an ear-wearable electronic device illustrated in FIG. 5;

FIG. 7 illustrates a respiration sensor deployed in an ear-wearable electronic device in accordance with any of the embodiments disclosed herein;

FIG. 8 illustrates a respiration sensor deployed in an ear-wearable electronic device in accordance with any of the embodiments disclosed herein;

FIG. 9 illustrates a respiration sensor deployed in an ear-wearable electronic device in accordance with any of the embodiments disclosed herein;

FIG. 10 illustrates additional processing details implemented by the PPG sensing circuitry and the motion sensing circuitry shown in previous figures;

FIG. 11 illustrates a method of selecting the best window of time from a sampling of PPG data and/or motion sensor data in accordance with any of the embodiments disclosed herein;

FIG. 12 illustrates a method of applying a sinus-fitting algorithm on PPG data and/or motion sensor data in accordance with any of the embodiments disclosed herein;

FIG. 13 illustrates a method for activating a respiration rate sensor of an ear-wearable electronic device in accordance with any of the embodiments disclosed herein; and

FIG. 14 illustrates a system involving the generation and distribution of respiration rate data by an ear-wearable electronic device in accordance with any of the embodiments disclosed herein.

The figures are not necessarily to scale. Like numbers used in the figures refer to like components. However, it will be understood that the use of a number to refer to a component in a given figure is not intended to limit the component in another figure labeled with the same number.

### DETAILED DESCRIPTION

Valuable health information can be derived from measurement of a person's respiration rate. Provision of a respiration rate sensing facility in an ear-wearable electronic device can improve the accuracy of disease detection as well as open new doors to many more feature possibilities, including emotional stress monitoring and cardiac arrythmia detection. Additionally, an ear-wearable electronic device which incorporates a respiration rate sensing facility according to the present disclosure can assist in tracking the progression of an illness, such as respiratory diseases. By tracking respiration rate and detecting increases in respiration rate overtime (e.g., via trend analyses), the worsening of respiratory disease can be accurately detected and monitored.

Some embodiments are directed to an ear-wearable electronic device which incorporates a PPG-based sensing facility configured to generate an estimate of a wearer's respiration rate. Some embodiments are directed to an ear-wearable electronic device which incorporates a motion-based sensing facility configured to generate an estimate of the wearer's respiration rate. Some embodiments are directed to an ear-wearable electronic device which incorporates a PPG-based sensing facility and a motion-based sensing facility configured to generate an estimate of the wearer's respiration rate.

Respiration induces variations in a photoplethysmogram in three different ways. (1) Respiratory-Induced Intensity Variation (RIIV): involves changes in venous return due to changes in intra-thoracic pressure throughout the respiratory cycle, which cause a baseline (DC) modulation of the PPG signal. (2) Respiratory-Induced Amplitude Variation (RIAV): during inspiration, left ventricular stroke volume decreases due to changes in intra-thoracic pressure leading to the decreased pulse amplitude. The opposite occurs during expiration. (3) Respiratory-Induced Frequency Variation (RIFV): heart rate varies throughout the respiratory cycle. Heart rate increases during inspiration and decreases during expiration. This phenomenon, known as respiratory sinus arrhythmia (RSA), is mainly due to the autonomic regulation of HR during respiration.

Respiration induces variations in a motion sensor signal. During respiration, the body moves according to the breathing rate of the subject. For example, the chest wall expands and contracts in response to inhalation and exhalation during breathing. By analyzing these motions, measured by a motions sensor (e.g., an accelerometer), respiration rate can be estimated.

Embodiments of the disclosure are defined in the claims. However, below there is provided a non-exhaustive listing of non-limiting examples. Any one or more of the features of these examples may be combined with any one or more features of another example, embodiment, or aspect described herein.

Example Ex1. A method of determining respiration rate using an ear-wearable electronic device comprises obtaining motion information from a motion sensor of the ear-wearable electronic device, generating a first respiration rate estimate using the motion information, obtaining photoplethysmographic (PPG) data from a PPG sensor of the ear-wearable electronic device, generating a second respiration rate estimate using the PPG data, and producing a respiration rate estimate using the first and second respiration rate estimates.

Example Ex2. The method according to Ex1, wherein generating the first respiration rate estimate comprises filtering the motion information using a bandpass filter configured to pass frequencies in a frequency range consistent with human breathing, and applying a sinus fitting to the bandpass-filtered motion information to generate the first respiration rate estimate.

Example Ex3. The method according to Ex1 or Ex2, wherein generating the second respiration rate estimate comprises filtering the PPG data using a high pass filter having a specified cutoff frequency, performing a time domain-to-frequency domain transform on the high pass-filtered PPG data, performing peak and local minimum detection on the transformed PPG data, and applying a sinus fitting to heights of peaks of the transformed PPG data to generate the second respiration rate estimate.

Example Ex4. The method according to one or more of Ex1 to Ex3, comprising capturing the motion information in a plurality of temporally spaced first windows, capturing the PPG data in a plurality of temporally spaced second windows, and selecting one of the first windows and one of the second windows for processing based on predefined spectral content criteria.

Example Ex5. The method of Ex4, wherein the predefined spectral content criteria comprises a highest peak in a spectral domain in the range of about 0.1 Hz to about 0.5 Hz. Example Ex6. The method according to one or more of Ex1 to Ex5, comprising performing a test of motion sensor signal integrity, and performing a test of PPG sensor signal integrity.

Example Ex7. The method according to one or more of Ex1 to Ex6, wherein producing the respiration rate estimate comprises processing the first and second first respiration rate estimates using a fusion algorithm to produce the respiration rate estimate.

Example Ex8. The method according to one or more of Ex1 to Ex7, wherein producing the respiration rate estimate comprises comparing the second respiration rate to a threshold, and outputting the second respiration rate estimate in response to the second respiration rate exceeding the threshold.

Example Ex9. The method according to one or more of Ex1 to Ex8, wherein producing the respiration rate estimate comprises comparing the second respiration rate to a threshold, and outputting the first respiration rate estimate in response to the second respiration rate failing to exceed the threshold.

Example Ex10. The method according to one or more of Ex1 to Ex9, comprising, performing an activity status test using the motion information and PPG data, and determining if a measure of the wearer's physical activity based on the motion information is consistent with the wearer's respiration rate estimated using the PPG data.

Example Ex11. The method according to one or more of Ex1 to Ex10, comprising performing a validity test of the second respiration rate estimate by comparing the second respiration rate estimate to a threshold.

Example Ex12. The method according to one or more of Ex1 to Ex11, comprising calculating the respiration rate estimate of the wearer in response to successful signal integrity, activity, and validity tests.

Example Ex13. The method according to one or more of Ex1 to Ex12, comprising communicating respiration rate data alone or in combination with other physiologic data to one or both of an external electronic device and a cloud database.

Example Ex14. The method according to Ex13, comprising generating one or more of an early warning score, long term analyses, and respiration rate trending reports by one or both of the external electronic device or a cloud processor.

Example Ex15. An ear-wearable electronic device, comprises a motion sensor configured to generate motion information and a first respiration rate estimated using the motion information, a photoplethysmographic (PPG) sensor configured to generate PPG data and a second respiration rate estimate using the PPG data, a processor configured to produce a respiration rate estimate using the first and second respiration rate estimates, and a communication device configured to communicate the respiration rate estimate to one or both of an external electronic device and a cloud database.

Example Ex16. The device according to Ex15, comprising bandpass filter configured to pass frequencies of the motion information in a frequency range consistent with human breathing, and a sinus fitting module configured to apply sinus fitting to the bandpass-filtered motion information to generate the first respiration rate estimate.

Example Ex17. The device according to Ex15 or Ex16, wherein generating the second respiration rate estimate comprises a high pass filter having a specified cutoff frequency configured to filter the PPG data, a peak and local minimum detector configured to perform peak and local minimum detection on frequency transformed PPG data, and a sinus fitting module configured to apply a sinus fitting to heights of peaks of the frequency transformed PPG data to generate the second respiration rate estimate.

Example Ex18. The device according to one or more of Ex15 to Ex17, comprising a memory configured to capture the motion information in a plurality of temporally spaced first windows and to capture the PPG data in a plurality of temporally spaced second windows, wherein the processor is configured to select one of the first windows and one of the second windows for processing based on predefined spectral content criteria.

Example Ex19. The device of Ex18, wherein the predefined spectral content criteria comprises a highest peak in a spectral domain in the range of about 0.1 Hz to about 0.5 Hz.

Example Ex20. The device according to one or more of Ex15 to Ex19, wherein the processor is configured to perform a test of motion sensor signal integrity, and perform a test of PPG sensor signal integrity.

Example Ex21. The device according to one or more of Ex15 to Ex20, comprising a data fusion module configured to process the first and second first respiration rate estimates using a fusion algorithm to produce the respiration rate estimate.

Example Ex22. The device according to one or more of Ex15 to Ex21, wherein the data fusion module is configured to compare the second respiration rate to a threshold, and output the second respiration rate estimate in response to the second respiration rate exceeding the threshold.

Example Ex23. The device according to one or more of Ex15 to Ex22, wherein the data fusion module is configured to compare the second respiration rate to a threshold, and output the first respiration rate estimate in response to the second respiration rate failing to exceed the threshold.

Example Ex24. The device according to one or more of Ex15 to Ex23, wherein the processor is configured to perform an activity status test using the motion information and PPG data, and determine if a measure of the wearer's physical activity based on the motion information is consistent with the wearer's respiration rate estimated using the PPG data.

Example Ex25. The device according to one or more of Ex15 to Ex24, wherein the processor is configured to perform a validity test of the second respiration rate estimate by comparing the second respiration rate estimate to a threshold.

Example Ex26. The device according to one or more of Ex15 to Ex25, wherein the processor is configured to calculate the respiration rate estimate of the wearer in response to successful signal integrity, activity, and validity tests.

Example Ex27. The device according to one or more of Ex15 to Ex26, wherein the processor is configured to communicate respiration rate data alone or in combination with other physiologic data to one or both of an external electronic device and a cloud database.

Example Ex28. The device according to Ex27, wherein one or both of the external electronic device and a cloud processor are configured to generate one or more of an early warning score, long term analyses, and respiration rate trending reports.

Example Ex29. A method of determining respiration rate using an ear-wearable electronic device comprises obtaining motion information indicative of body motion from a motion sensor of the ear-wearable electronic device, generating, using a processor of the device, a respiration rate estimate using the motion information, and communicating the respiration rate estimate to one or both of an external electronic device and a cloud database.

Example Ex30. An ear-wearable electronic device comprises a motion sensor configured to generate motion information indicative of body motion, a processor configured to produce a respiration rate estimate using the motion information, and a communication device configured to communicate the respiration rate estimate to one or both of an external electronic device and a cloud database.

Example Ex31. A method of determining respiration rate using an ear-wearable electronic device comprises obtaining photoplethysmographic (PPG) data from a PPG sensor of the ear-wearable electronic device, generating, using a processor of the device, a respiration rate estimate using the PPG data, and communicating the respiration rate estimate to one or both of an external electronic device and a cloud database.

Example Ex32. An ear-wearable electronic device comprises a photoplethysmographic (PPG) sensor configured to generate PPG data, a processor configured to produce a respiration rate estimate using the PPG data, and a communication device configured to communicate the respiration rate estimate to one or both of an external electronic device and a cloud database.

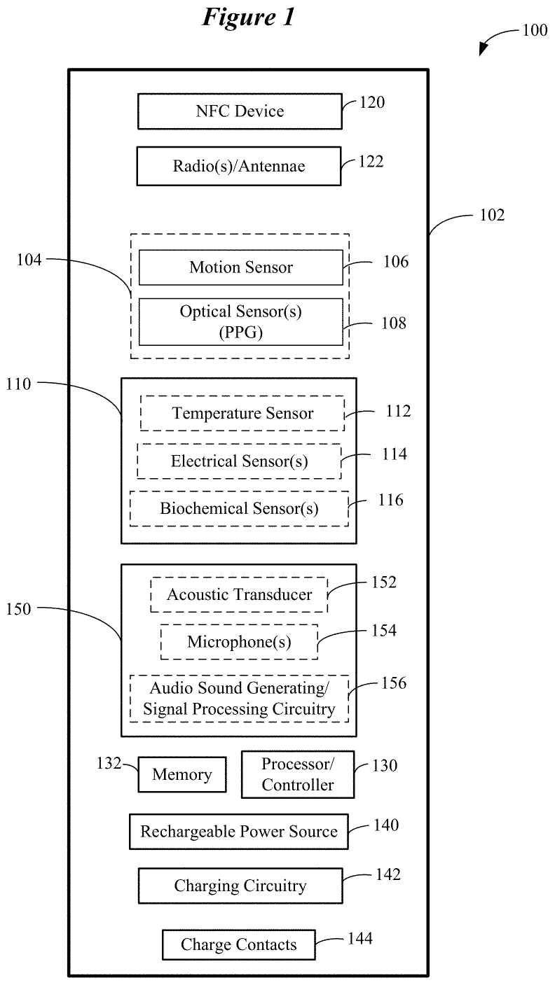

FIG. 1 is a block diagram of a representative ear-wearable electronic device 100 which incorporates a respiration sensor in accordance with any of the embodiments disclosed herein. The device 100 is representative of a wide variety of electronic devices configured to be deployed in, on or about an ear of a wearer, including any of the devices discussed herein. In some implementations, the device 100 is configured as a hearable in which amplified sound is communicated one or both ears of a wearer. In other implementations, the device 100 is configured as a physiologic or biometric monitoring device, which may include or exclude sound amplification circuitry. The device 100 can be configured to include or exclude some of the representative components shown in FIG. 1.

The term ear-wearable electronic device 100 of the present disclosure refers to a wide variety of ear-wearable electronic devices that can aid a person with impaired hearing. The term ear-wearable electronic device also refers to a wide variety of devices that can produce optimized or processed sound for persons with normal hearing. Ear-wearable electronic devices of the present disclosure include hearables (e.g., earbuds) and hearing aids (e.g., hearing instruments), for example. Ear-wearable electronic devices include, but are not limited to ITE, ITC, CIC or IIC type hearing devices or some combination of the above. Some ear-wearable electronic devices can be devoid of an audio processing facility, and be configured as an ear-wearable biometric sensor (e.g., a respiration sensor alone or in combination with any of the other physiologic and/or motion sensors disclosed herein). In this disclosure, reference is made to an “ear-wearable electronic device,” which is understood to refer to a system comprising a single ear device (left or right) or both a left ear device and a right ear device.

According to any of the embodiments disclosed herein, the device 100 includes a housing 102 configured for deployment in an ear canal of a wearer. In some implementations, the housing 102 includes a shell having a uniquely-shaped outer surface (e.g., an organic shape) that corresponds uniquely to an ear geometry of a wearer of the device (e.g., a custom RIC in-ear device). In other implementations, the shell of the housing 102 has a generic or standard shape having an outer surface configured for deployment in ear canals of a population of wearers.

The device 100 includes a respiration sensor 104 configured to sense physiologic activity from which a respiration rate estimate is calculated by the device 100. In some implementations, the respiration sensor 104 includes a motion sensor 106, such as an accelerometer (e.g., a 3-axis accelerometer). In other implementations, the respiration sensor 104 includes one or more optical sensors 108, such as a PPG sensor. In some implementations, the respiration sensor 104 includes a motion sensor 106 and a PPG sensor.

In implementations in which the respiration sensor 104 includes a PPG sensor 108, the shell of the housing 102 includes a window through a proximal portion of the shell and positioned at a tragal wall when the device is deployed in the wearer's ear. The PPG sensor 108 is situated within the window of the shell. The shell can include a seal arrangement comprising an acoustic seal to inhibit ambient sound from reaching the wearer's eardrum. The seal arrangement can also include a light seal configured to inhibit ambient light from reaching the PPG sensor 108. The PPG sensor 108 is preferably situated in the window such that no air gaps exist between the PPG sensor 108 and tissue of the ear canal when deployed in the wearer's ear. The combination of a light seal and prevention of air gaps provides for signals produced by the PPG sensor 108 having a high signal-to-noise ratio.

In some implementations (e.g., physiologic or biometric monitoring configurations), the device 100 can include a physiologic sensor facility 110 which can include one or more additional physiologic sensors. For example, the device 100 can include one or more temperature sensors 112 configured to produce signals from which an estimate of a wearer's core body temperature can be calculated by the device 100. The temperature sensor or sensors (e.g., a proximal temperature sensor and a distal temperature sensor) can be thermistors. Various types of thermistors can be incorporated into the ear-wearable electronic devices of the present disclosure (e.g., those having a negative temperature coefficient (e.g., a negative temperature coefficient (NTC) chip) and those having a positive temperature coefficient (PTC)). In some implementations, the temperature sensor(s) 112 can be implemented as a SMD (surface mount device) thermistor, a thermocouple, a resistance temperature detector (RTD), a digital thermistor, or other type of resistance temperature sensors.

The device 100 can include one or more physiologic electrode-based sensors 114, such as an ECG, oxygen saturation (SpO2), respiration, EMG, EEG, EOG, galvanic skin response, and/or electrodermal activity sensor. The device 100 can include one or more biochemical sensors 116 (e.g., glucose concentration, PH value, Ca+ concentration, hydration). Embodiments disclosed herein can incorporate one or more of the sensors disclosed in commonly-owned co-pending U.S. Patent Application Ser. Nos. 63/125,700 filed Dec. 15, 2020 under Attorney Docket No. ST0922PRV/0532.000922US60 and 63/126,426 filed Dec. 16, 2020 under Attorney Docket No. ST0931PRV/0532.000931US60, both of which are incorporated herein by reference in their entireties.

The device 100 can include an NFC device 120 (e.g., a NFMI device), and, additionally or alternatively, may include one or more RF radios/antennae 122 (e.g., compliant with a Bluetooth® or IEEE 802.11 protocol). The RF radios/antennae 122 can be configured to effect communications with an external electronic device, communication system, and/or the cloud. Data acquired by the ear-wearable electronic device 100 can be communicated to an external electronic device, such as a smartphone, laptop, network server, and/or the cloud (e.g., a cloud server/database and/or processor). The device 100 typically includes a rechargeable power source 140 (e.g., a lithium-ion battery) operably coupled to charging circuitry 142 and charge contacts 144. The device 100 includes a processor (e.g., a controller) 130 coupled to memory 132.

Among other duties, the processor 130 is configured to calculate an estimated respiration rate of the device wearer in accordance with any of the embodiments disclosed herein. The processor 130 is also configured to calculate other biometric or physiologic parameters or conditions using the various sensor signals discussed herein.

In accordance with any of the embodiments disclosed herein, the device 100 can be configured as a hearing device or a hearable which includes an audio processing facility 150. The audio processing facility 150 includes sound generating circuitry and can also include audio signal processing circuitry 156 coupled to an acoustic transducer 152 (e.g., a sound generator, speaker, receiver, bone conduction device). In some implementations, the audio processing facility 150 includes one or more microphones 154 coupled to the audio signal processing circuitry 156. In other implementations, the device 100 can be devoid of the one or more microphones 154. In further implementations, the device 100 can be devoid of the audio processing facility 150, and be configured as an ear-wearable biometric sensor (e.g., a respiration sensor alone or in combination with any of the other sensors disclosed herein).

According to implementations that incorporate the audio processing facility 150, the device 100 can be implemented as a hearing assistance device that can aid a person with impaired hearing. For example, the device 100 can be implemented as a monaural hearing aid or a pair of devices 100 can be implemented as a binaural hearing aid system, in which case left and right devices 100 are deployable with corresponding left and right wearable sensor units. The monaural device 100 or a pair of devices 100 can be configured to effect bi-directional communication (e.g., wireless communication) of data with an external source, such as a remote server via the Internet or other communication infrastructure. The device or devices 100 can be configured to receive streaming audio (e.g., digital audio data or files) from an electronic or digital source. Representative electronic/digital sources (e.g., accessory devices) include an assistive listening system, a streaming device (e.g., a TV streamer or audio streamer), a remote microphone, a radio, a smartphone, a laptop, a cell phone/entertainment device or other electronic device that serves as a source of digital audio data, control and/or settings data or commands, and/or other types of data files.

The processor/controller 130 shown in FIG. 1 can include one or more processors or other logic devices. For example, the processor/controller 130 can be representative of any combination of one or more logic devices (e.g., multi-core processor, digital signal processor (DSP), microprocessor, programmable controller, general-purpose processor, special-purpose processor, hardware controller, software controller, a combined hardware and software device) and/or other digital logic circuitry (e.g., ASICs, FPGAs), and software/firmware configured to implement the functionality disclosed herein. The processor/controller 130 can incorporate or be coupled to various analog components (e.g., analog front-end), ADC and DAC components, and Filters (e.g., FIR filter, Kalman filter). The processor/controller 130 can incorporate or be coupled to memory 132 as previously discussed. The memory 132 can include one or more types of memory, including ROM, RAM, SDRAM, NVRAM, EEPROM, and FLASH, for example.

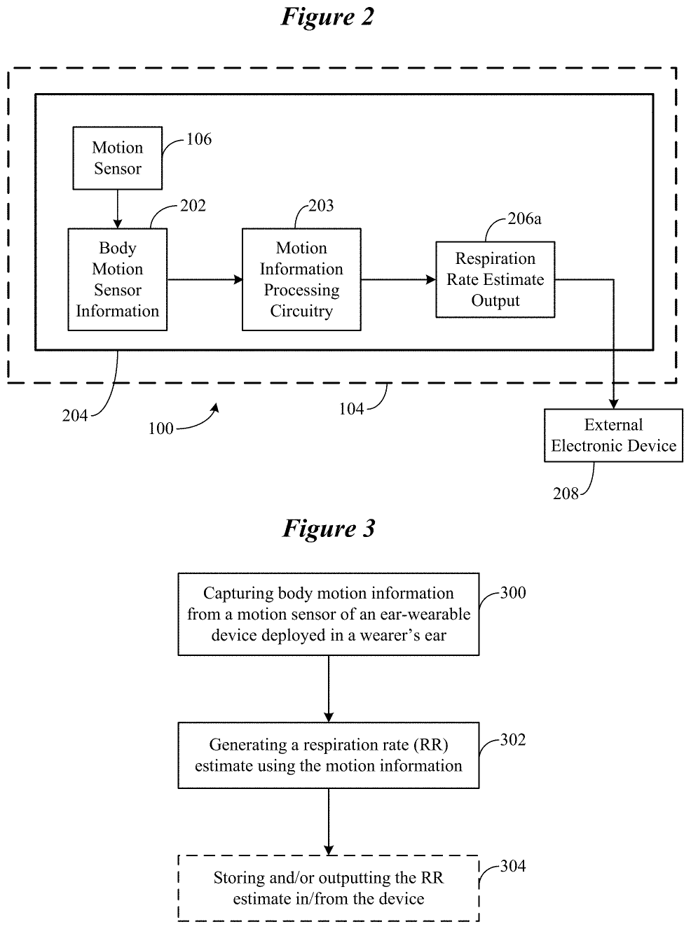

FIG. 2 illustrates a respiration sensor 104 integral to an ear-wearable electronic device 100 in accordance with any of the embodiments disclosed herein. The respiration sensor 104 illustrated in FIG. 2 includes motion sensing circuitry 204 comprising a motion sensor 106. The motion sensor 106 can be or include an accelerometer (e.g., a 3-axis accelerometer), a gyroscope, an inertial measurement unit (IMU), a magnetometer or any combination of these motion sensing devices. The motion sensor 106 is configured to produce body motion sensor information 202 responsive to movement of a wearer's body. For example, the motion sensor 106 can be configured to be sensitive to chest wall motion associated with inhalation and exhalation during breathing by the wearer. The body motion sensor information 202 is communicated to motion information processing circuitry 203, which may be integral in whole or in part to the processor 130 or to a separate processor or digital logic circuitry. The motion information processing circuitry 203 is configured to generate an estimate of respiration rate using the body motion sensor information 202. The respiration rate estimate is communicated to an output 206a of the motion sensing circuitry 204. The respiration rate estimate output 206a can be communicated to an external electronic device 208 (e.g., via a communication device of the ear-wearable electronic device 100) and/or stored in a memory of the ear-wearable electronic device 100.

FIG. 3 illustrates a method implemented by the ear-wearable electronic device 100 illustrated in FIG. 2. The method shown in FIG. 3 involves capturing 300 body motion information from a motion sensor of an ear-wearable electronic device deployed in the wearer's ear. The method also involves generating 302 a respiration rate estimate using the motion information. The method may also involves storing and/or outputting 304 the respiration rate estimate in/from the ear-wearable electronic device.

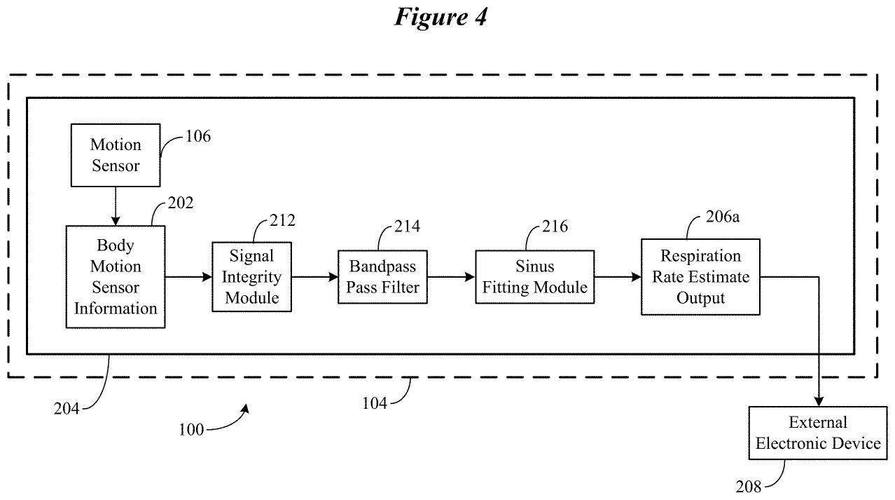

FIG. 4 illustrates a respiration sensor 104 deployed in an ear-wearable electronic device 100 in accordance with any of the embodiments disclosed herein. The respiration sensor 104 illustrated in FIG. 4 shows additional details of the motion sensing circuitry 204 shown in FIG. 3. The motion sensing circuitry 204 includes a motion sensor 106 of a type previously described which is configured to produce body motion sensor information 202 in a manner previously described.

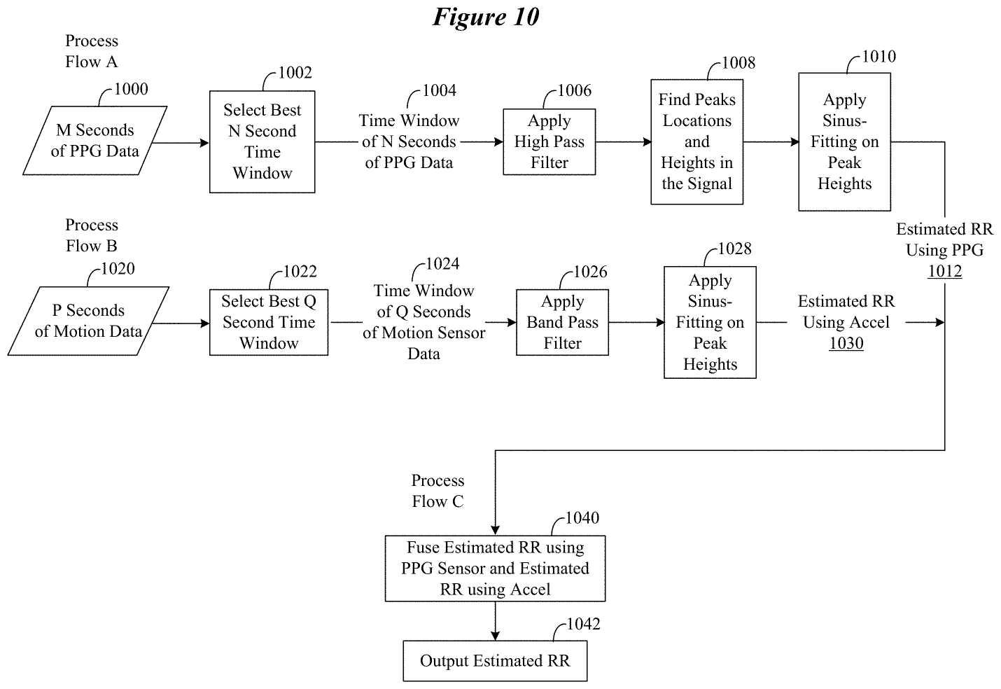

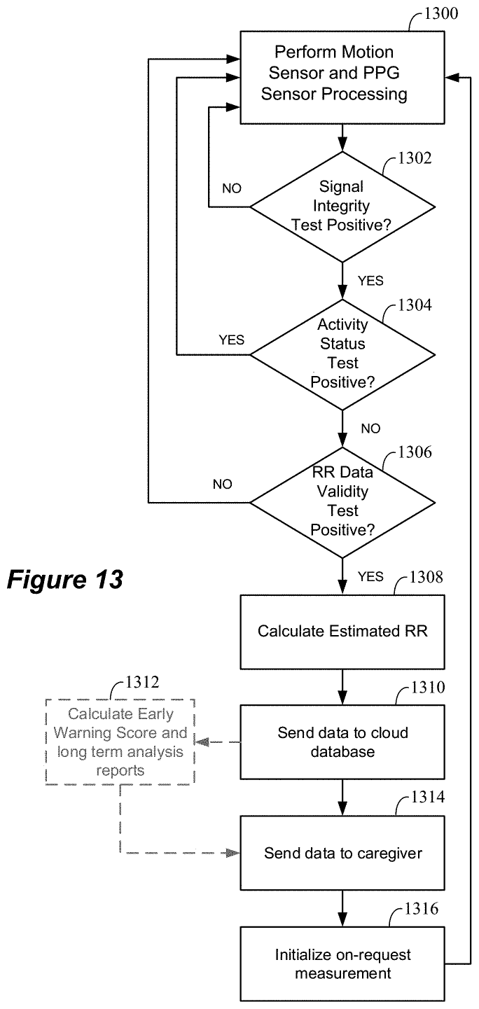

As shown in FIG. 4, the body motion sensor information 202 is communicated to a signal integrity module 212 which is configured to perform a signal integrity test using various metrics, details of which will be described with reference to FIG. 13. A selected time segment of the body motion sensor information 202 is communicated to a bandpass filter 214 configured to pass sensor information 202 within a frequency range consistent with human breathing. For example, the bandpass filter 214 can be configured to pass frequencies in the range of about 0.15 Hz to about 0.5 Hz. The time segment of the body motion sensor information 202 is selected based on a time window selection algorithm, details of which are shown in FIG. 10. The bandpass-filtered body motion sensor information 202 is communicated to a sinus fitting module 216 configured to perform a least-squares fitting operation, details of which will be described with reference to FIG. 10. The sinus fitting module 216 generates a respiration rate estimate output 206a which can be stored internally within the ear-wearable electronic device 100 and/or communicated to an external electronic device 208.

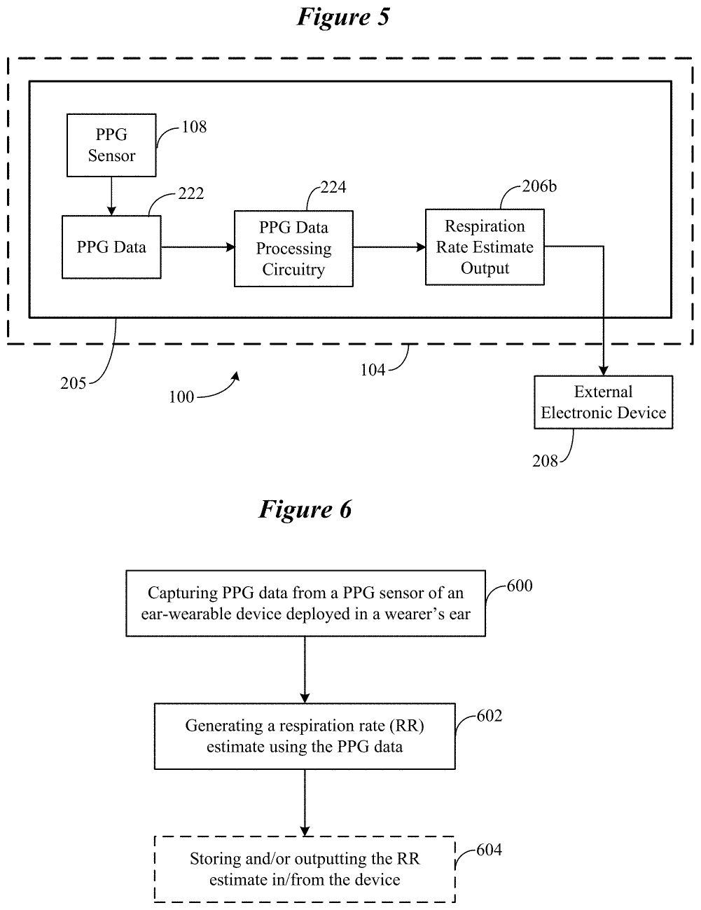

FIG. 5 illustrates a respiration sensor 104 integral to an ear-wearable electronic device 100 in accordance with any of the embodiments disclosed herein. The respiration sensor 104 illustrated in FIG. 5 includes PPG sensing circuitry 205 comprising a PPG sensor 108. The PPG sensor 108 may be implemented as a pulse oximeter or other form of sensor capable of optically obtaining a plethysmogram. The PPG sensor 108 is configured to produce PPG data 222 from within the wearer's ear. For example, the PPG sensor 108 can be situated in a window of the housing 102 of the ear-wearable electronic device 100 such that the PPG sensor 108 faces the tragal wall when the device 100 is fully deployed in the wearer's ear. As was previously discussed, the shell of the device housing can include a seal arrangement comprising an acoustic seal and a light seal, wherein the light seal is configured to inhibit ambient light from reaching the PPG sensor 108. The PPG sensor is situated in the window of the shell so that no air gaps exist between the PPG sensor 108 and tissue of the wearer's tragal wall when the device 100 is deployed in the wearer's ear.

PPG data 222 produced by the PPG sensor 108 is communicated to PPG data processing circuitry 224, which may be integral in whole or in part to the processor 130 or to a separate processor or digital logic circuitry. The PPG data processing circuitry 224 is configured to generate an estimate of respiration rate using the PPG data 222. The respiration rate estimate is communicated to an output 206b of the PPG sensing circuitry 205. The respiration rate estimate output 206b can be communicated to an external electronic device 208 (e.g., via a communication device of the ear-wearable electronic device 100) and/or stored in a memory 132 of the ear-wearable electronic device 100.

FIG. 6 illustrates a method implemented by an ear-wearable electronic device 100 illustrated in FIG. 5. The method shown in FIG. 6 involves capturing 600 PPG data from a PPG sensor of an ear-wearable electronic device deployed in the wearer's ear. The method also involves generating 603 a respiration rate estimate using the PPG data. The method may also involve storing and/or outputting 604 the respiration rate estimate in/from the ear wearable electronic device.

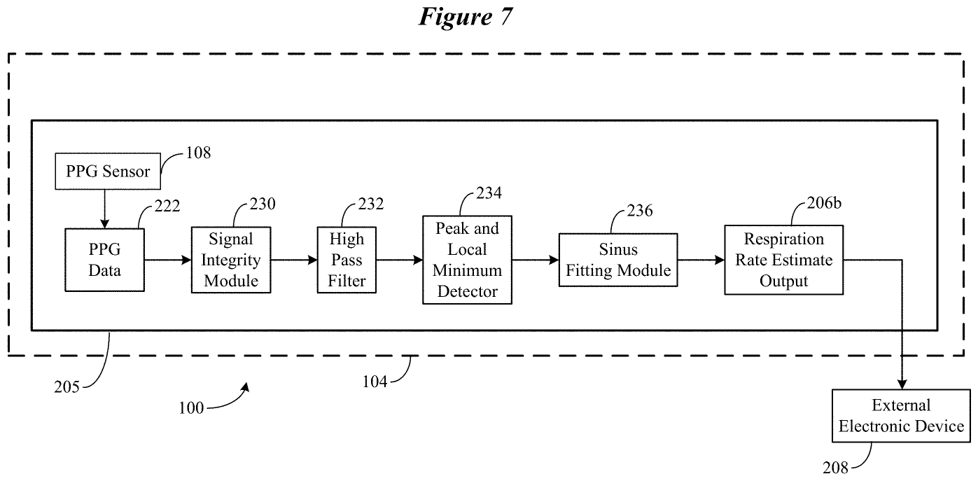

FIG. 7 illustrates a respiration sensor 104 deployed in an ear-wearable electronic device 100 in accordance with any of the embodiments disclosed herein. The respiration sensor 104 illustrated in FIG. 7 shows additional details of the PPG sensing circuitry 205. The PPG data 222 produced by the PPG sensor 108 is communicated to a signal integrity module 230 which is configured to perform a signal integrity test using various metrics, details of which will be described with reference to FIG. 13. A selected time segment of the PPG data 222 is communicated to a high pass filter 232 configured to remove noise that appears in low frequencies that are irrelevant for the respiration rate estimation. For example, the high pass filter 232 can be configured to pass frequencies above about 0.3 Hz (e.g., f_{corner}=0.3 Hz). The high pass-filtered PPG data 222 is communicated to a peak and local minimum detector 234, which is configured to analyze the amplitude variations of the high pass-filtered PPG data 222. The high pass-filtered PPG data 222 is communicated to a sinus fitting module 236 configured to perform a least-squares fitting operation, details of which will be described with reference to FIG. 10. The sinus fitting module 236 generates a respiration rate estimate output 206b which can be stored internally within the memory 132 of the ear-wearable electronic device 100 and/or communicated to an external electronic device 208.

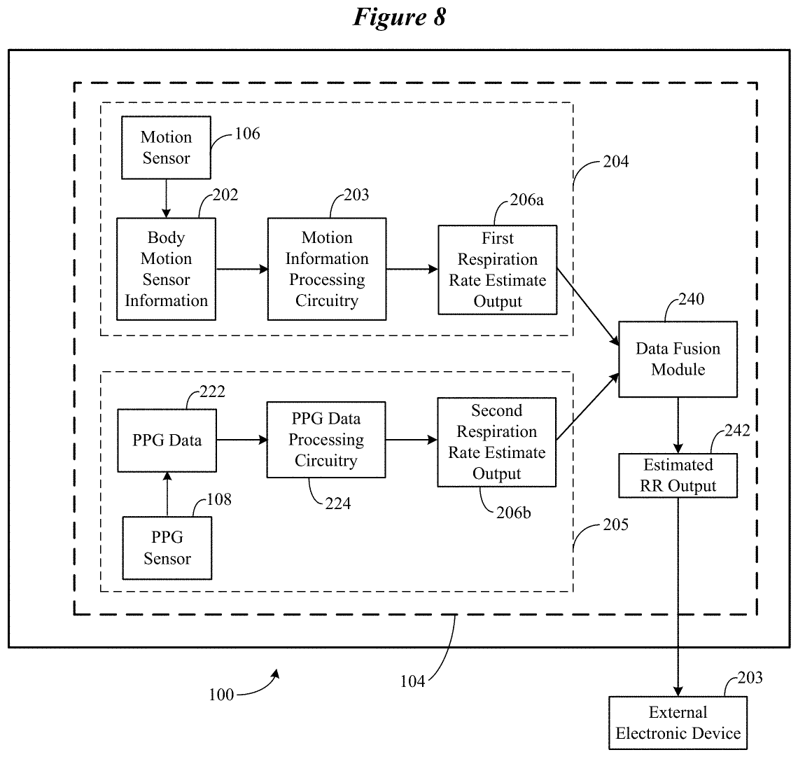

FIG. 8 illustrates a respiration sensor 104 deployed in an ear-wearable electronic device 100 in accordance with any of the embodiments disclosed herein. The respiration sensor 104 shown in FIG. 8 includes motion sensing circuitry 204 and PPG sensing circuitry 205. The components and functionality of the motion sensing circuitry 204 are equivalent to that described previously with reference to FIG. 2. The components and functionality of the PPG sensing circuitry 205 are equivalent to that described previously with reference to FIG. 5. The motion sensing circuitry 204 produces a first respiration rate estimate output 206a which is communicated to a data fusion module 240. The PPG sensing circuitry 205 produces a second respiration rate estimate output 206b which is communicated to the data fusion module 240. The data fusion module 240 is configured to process the first and second respiration rate outputs 206a, 206b to produce an estimated respiration output 242 (see, e.g., FIG. 10). The estimated respiration output 242 can be stored internally within the memory 132 of the ear-wearable electronic device 100 and/or communicated to an external electronic device 208.

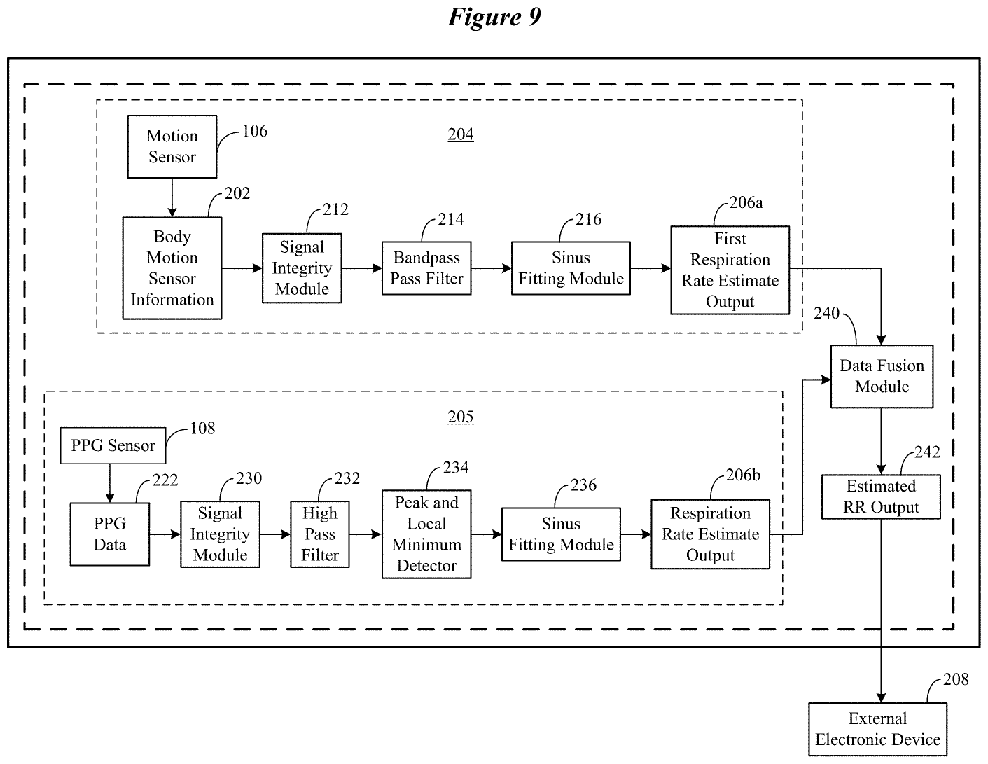

FIG. 9 illustrates a respiration sensor 104 deployed in an ear-wearable electronic device 100 in accordance with any of the embodiments disclosed herein. Respiration sensor 104 shown in FIG. 9 includes motion sensing circuitry 204 and PPG sensing circuitry 205.

The components and functionality of the motion sensing circuitry 204 are equivalent to that described previously with reference to FIG. 4. The components and functionality of the PPG sensing circuitry 205 are equivalent to that previously described with reference to FIG. 7. The motion sensing circuitry 204 produces a first respiration rate estimate output 206a which is communicated to a data fusion module 240. The PPG sensing circuitry 205 produces a second respiration rate estimate output 206b which is communicated to the data fusion module 240. The data fusion module 240 is configured to process the first and second respiration rate outputs 206a, 206b to produce an estimated respiration output 242. The estimated respiration output 242 can be stored internally within the memory 132 of the ear-wearable electronic device 100 and/or communicated to an external electronic device 208.

FIG. 10 illustrates additional processing details implemented by the PPG sensing circuitry 205 and the motion sensing circuitry 204 shown in previous figures. Process flow A involves the processing of M seconds (e.g., 60 seconds) of PPG data 1000 by the PPG sensing circuitry 205. Process flow A also involves selection 1002 of the best (e.g., most useful or suitable) N seconds (e.g., 20 seconds) of the M seconds of PPG data 1000, the result of which is a time window 1004 of N seconds of PPG data. The N seconds of PPG data 1004 is applied to a high pass filter 1006 having a specified corner frequency (e.g., f_{corner}=0.3 Hz). The locations and heights of the peaks in the high pass-filtered PPG data are identified, such as by using a peak and local minimum detector. Process flow A concludes by applying a sinus-fitting algorithm (e.g., least-squares fitting) on the heights of the peaks, from which an estimated respiration rate 1012 using the PPG data is computed.

Process flow B involves the processing of P seconds (e.g., 60 seconds) of motion sensor data 1020 by the motion sensing circuitry 204. Process flow B also involves selection 1022 of the best (e.g., most useful or suitable) Q seconds (e.g., 20 seconds) of the P seconds of motion sensor data 1020, the result of which is a time window 1024 of Q seconds of motion data. The Q seconds of motion data 1024 is applied to a bandpass filter 1026 having a specified passband (e.g., from about 0.15 Hz to about 0.5 Hz). Process flow B concludes by applying a sinus-fitting algorithm (e.g., least-squares fitting) on the bandpass-filtered motion sensor data, from which an estimated respiration rate 1030 using the motion sensor data is computed.

Data fusion is performed on the estimated respiration rates 1012, 1030 to determine the output estimated respiration rate generated by the respiration sensor 104. Process flow C involves fusing 1040 the estimated respiration rate 1012 using the PPG data and the estimated respiration rate 1030 using the motion sensor to generate 1042 an output estimated respiration rate. According to some implementations, process flow C can involve a comparison of the estimated respiration rate using the PPG data to a threshold (e.g., 25). If the estimated respiration rate using the PPG data exceeds the threshold, the estimated respiration rate using the PPG sensor is output by the respiration sensor 104. If the estimated respiration rate using the PPG data does not exceed the threshold, the estimated respiration rate using the motion sensor is output by the respiration sensor 104. In other implementations, a fusing the estimated respiration rates 1012 and 1030 can involve weighting each of the estimated respiration rate 1012 using the PPG data and the estimated respiration rate 1030 using the motion sensor to generate 1042 an output estimated respiration rate. Weighting can be based on a number of factors, including, for example, the availability and/or relative integrity of the PPG and motion sensor data.

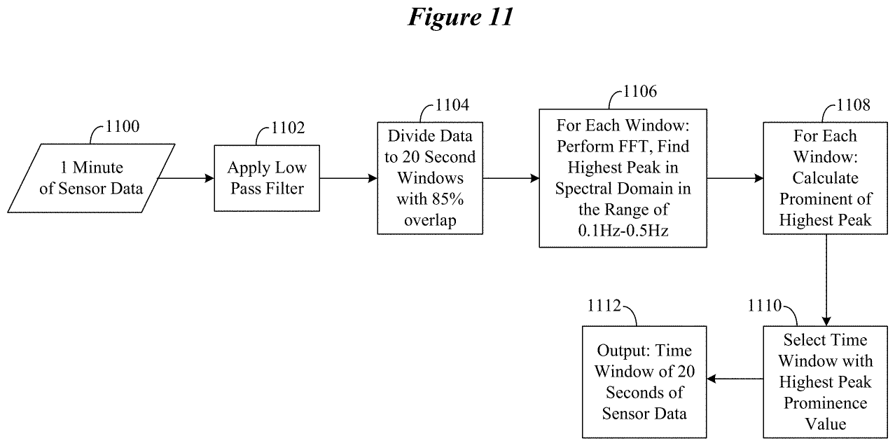

FIG. 11 illustrates a method of selecting the best (e.g., most useful or suitable) window of time from a sampling of PPG data and/or motion sensor data in accordance with any of the embodiments disclosed herein. In the representative examples shown in FIG. 11, one minute of sensor data is acquired 1100. It is understood that the amount of sensor data acquired in block 1100 can be longer or shorter than one minute. This sensor data is applied 1102 to a low pass filter having a specified corner frequency (e.g., f_{corner}=0.5 Hz). The method involves dividing 1104 the low pass-filtered sensor data into 20 second windows with an 85% overlap. It is understood that the amount of low-pass filtered sensor data acquired in block 1104 and the extent of the overlap can be longer or shorter than shown in FIG. 11.

In block 1106, for each of the 20 second windows, a fast Fourier transform (FFT) is performed on the data, and the highest peak in the spectral domain in the range of 0.1 Hz to 0.5 Hz is determined. In block 1108, for each of the 20 second windows, the prominence of the highest peak is calculated. The time window having the highest peak prominence value is selected 1110, resulting in the output 1112 of the selected time window of 20 seconds of sensor data.

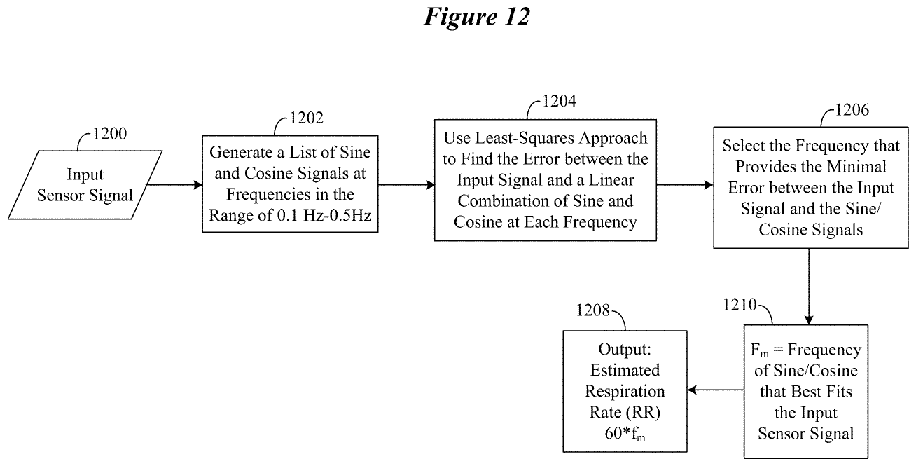

FIG. 12 illustrates a method of applying a sinus-fitting algorithm on PPG data and/or motion sensor data in accordance with any of the embodiments disclosed herein. The method shown in FIG. 12 begins with the input 1200 of a PPG or motion sensor signal, from which a list of sine and cosine signals at frequencies in the range of 0.1 Hz to 0.5 Hz is generated 1202. The method involves using 1204 a least-squares approach to find the error between the input signal and a linear combination of sine and cosine at each of the frequencies. The method also involves selecting 1206 the frequency that provides the minimal error between the input sensor signal and the sine/cosine signals. This results in selection 1210 of the frequency, Fₘ, of the sine/cosine list that best fits the input sensor signal. The method further involves the output 1208 of the estimated respiration rate using the equation RR=60*fₘ.

FIG. 13 illustrates a method for activating a respiration rate sensor of an ear-wearable electronic device in accordance with any of the embodiments disclosed herein. The method shown in FIG. 13 involves criteria for respiration rate algorithm activation. The method shown in FIG. 13 involves performing motion sensor and PPG sensor processing 1300, which involves activating the sensors and receiving sensor signals by a processor of the ear-wearable electronic device 100. The processing of motion and PPG sensor signals by the processor involves performing a signal integrity test. The signal integrity test involves the integrity of the motion and PPG sensor signals based on various calculations such as signal to noise ratio, skin contact integrity (e.g., resistance, perfusion index), and/or a correlation coefficient, R, for example.

If the signal integrity test is negative, as tested at block 1302, processing of motion and PPG sensor signals continues at block 1300. If the signal integrity test is positive, an activity status test is initiated. The activity status test can involve determining whether high respiration rate, if it exists, is caused by the wearer's current or prior activity. To be negative, both prior activity (e.g., in the prior 15-minutes period) and current activity need to be negative. For example, the activity status test can involve sensing of the wearer's level of physical activity using the motion sensor, and determining if the sensed level of physical activity is consistent with the wearer's respiration rate. Increased physical activity is associated with increased respiration rate, while decreased physical activity is associated with decreased respiration rate (e.g., within certain ranges). If the measure of physical activity is out of synchrony with the respiration rate (e.g., relative to predetermined ranges), then the activity status test may return a positive result.

If the activity status test is positive, as tested at block 1304, processing of motion and PPG sensor signals continues at block 1300. If the activity status test is negative, a respiration data validity test is initiated. The respiration rate validity (RRV) is calculated by the respiration algorithm implemented by the processor, based on the spectral content of the respiration rate signal. Criteria of the RRV calculation involves summing the energy level in all frequencies relevant for the RR estimation in the spectral domain. If the respiration rate validity test is not positive (e.g., negative), as tested at block 1306, processing of motion and PPG sensor signals continues at block 1300. If the respiration rate validity test is positive, processing continues at block 1308.

Assuming that all of the tests referenced in blocks 1302, 1304, and 1306 have been satisfied, the method continues by calculating the estimated respiration rate at block 1308. In some embodiments, the respiration rate data is communicated 1310 to a cloud database. It is noted that other physiologic data can be communicated to the cloud database, including heart rate, SpO2, temperature, electrodermal/skin contact data, and biochemical data (see physiologic sensors and parameters/conditions discussed with reference to FIG. 1.) A cloud processor can be configured to calculate 1312 an early warning score (e.g., poor respiration score relative to an acceptable respiration score) using the respiration rate data, perform long-term analysis (trends analyses), and generate various diagnostic reports. This and other data produced by the cloud processor can be communicated 1314 to the wearer or the wearer's caregiver via an external electronic device (e.g., a smartphone, tablet, laptop).

For example, the wearer and/or the caregiver can receive the wearer's daily average respiration rate and standard deviation in respiration rate. Long-term (e.g., trending) data analyses can include tracking changes in the wearer's respiration rate, and can also include correlating such changes with other measurable parameters (e.g., activity (e.g., before the measurement), human interaction, location, mood, etc.).

It is understood that, rather than or in addition to sending respiration data to the cloud database at block 1310, calculation of the early warning score, long-term analysis of the respiration data, and generation of diagnostic reports can be performed by an external electronic device operated by the wearer or the wearer's caregiver. In some implementations, an on-request (e.g. discretional) measurement can be initialized 1316 using an external electronic device operated by the wearer or the wearer's caregiver.

The method shown in FIG. 13 can be performed on a regular basis (e.g., hourly, every 4 hours, every 8 hours) and/or on a commanded on-request basis. For example, the operations shown in blocks 1300, 1302, 1304, and 1306 can be performed on a cyclical basis each N hours (e.g., N=1 or 2 hours). If, after M hours (e.g., M=8), the method does not progress to block 1308, the respiration rate sensor can be switched to a continuous mode of operation, and an alert can be generated. The alert can be communicated to an external electronic device, such as a smartphone operated by the wearer of the ear-wearable electronic device 100 or a caregiver (e.g., via the cloud). In addition, or alternatively, the alert can be communicated from the receiver or speaker of the ear-wearable electronic device 100 to the wearer's ear drum.

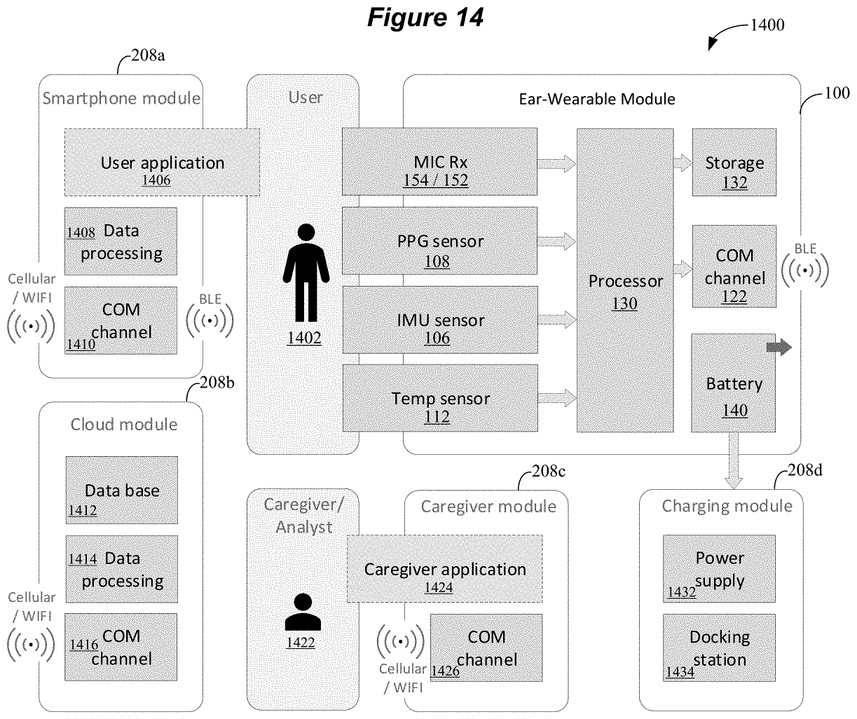

FIG. 14 illustrates a system involving the generation of respiration rate data by an ear-wearable electronic device in accordance with any of the embodiments disclosed herein. The system 1400 shown in FIG. 14 can be used implement the method shown in FIG. 13. The system 1400 includes the previously-described ear-wearable module 100, a smartphone module 208a, a cloud module 208b, a caregiver module 208c, and a charging module 208d. The ear-wearable module 100 includes a number of components such as a processor 130, memory 132, a communication device 122, a rechargeable battery 140, one or more microphones 154 and a receiver/speaker 152, a PPG sensor 108, a motion sensor 106, and, in some embodiments, a temperature sensor 112 (and/or other physiologic sensors disclosed herein). The ear-wearable electronic device 100 communicates with the smartphone module 208a via a communication channel 122, such as a BLE link.

The wearer 1402 of the ear-wearable electronic device 100 interacts with the device 100 via a user application 1406 supported by a processor and user interface of the smartphone module 208a. Smart phone module 208a includes a data processing facility 1408 and a communication device 1410, which can communicate with the communication device 122 of the ear-wearable electronic device 100 and may also include a cellular and/or Wi-Fi radio(s). The cloud module 208b includes a cloud database 1412, a data processing facility 1414 (e.g., a cloud processor), and a communication device 1416, such as a cellular and/or Wi-Fi radio(s).

The caregiver module 208c is configured to implement a caregiver application 1424 and includes a communication device 1426 (e.g., cellular and/or Wi-Fi radio(s)) configured to communicate with the cloud module 208b. A caregiver/analyst 1422 can interact with the smartphone module 208a and ear-wearable electronic device 100 in a manner described with reference to FIG. 13 (e.g., receiving and reviewing respiration rate data at block 1314, initiating an on-request measurement at block 1316). The charging module 208d includes a power supply 1432 and power management circuitry for charging the battery 140 of the ear-wearable electronic device 100. The charging module 208d can also include, or be configured as, a docking station 1434.

Although reference is made herein to the accompanying set of drawings that form part of this disclosure, one of at least ordinary skill in the art will appreciate that various adaptations and modifications of the embodiments described herein are within, or do not depart from, the scope of this disclosure. For example, aspects of the embodiments described herein may be combined in a variety of ways with each other. Therefore, it is to be understood that, within the scope of the appended claims, the claimed embodiments may be practiced other than as explicitly described herein.

All references and publications cited herein are expressly incorporated herein by reference in their entirety into this disclosure, except to the extent they may directly contradict this disclosure. Unless otherwise indicated, all numbers expressing feature sizes, amounts, and physical properties used in the specification and claims may be understood as being modified either by the term “exactly” or “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein or, for example, within typical ranges of experimental error.

The recitation of numerical ranges by endpoints includes all numbers subsumed within that range (e.g. 1 to 5 includes 1, 1.5, 2, 2.75, 3, 3.80, 4, and 5) and any range within that range. Herein, the terms “up to” or “no greater than” a number (e.g., up to 50) includes the number (e.g., 50), and the term “no less than” a number (e.g., no less than 5) includes the number (e.g., 5).

The terms “coupled” or “connected” refer to elements being attached to each other either directly (in direct contact with each other) or indirectly (having one or more elements between and attaching the two elements). Either term may be modified by “operatively” and “operably,” which may be used interchangeably, to describe that the coupling or connection is configured to allow the components to interact to carry out at least some functionality (for example, a radio chip may be operably coupled to an antenna element to provide a radio frequency electric signal for wireless communication).

Terms related to orientation, such as “top,” “bottom,” “side,” and “end,” are used to describe relative positions of components and are not meant to limit the orientation of the embodiments contemplated. For example, an embodiment described as having a “top” and “bottom” also encompasses embodiments thereof rotated in various directions unless the content clearly dictates otherwise.

Reference to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout are not necessarily referring to the same embodiment of the disclosure. Furthermore, the particular features, configurations, compositions, or characteristics may be combined in any suitable manner in one or more embodiments.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure.

As used in this specification and the appended claims, the singular forms “a,” “an,” and “the” encompass embodiments having plural referents, unless the content clearly dictates otherwise. As used in this specification and the appended claims, the term “or” is generally employed in its sense including “and/or” unless the content clearly dictates otherwise.

As used herein, “have,” “having,” “include,” “including,” “comprise,” “comprising” or the like are used in their open-ended sense, and generally mean “including, but not limited to.” It will be understood that “consisting essentially of” “consisting of,” and the like are subsumed in “comprising,” and the like. The term “and/or” means one or all of the listed elements or a combination of at least two of the listed elements.

The phrases “at least one of,” “comprises at least one of,” and “one or more of” followed by a list refers to any one of the items in the list and any combination of two or more items in the list.

What is claimed is:

1. A method of determining respiration rate using an ear-wearable electronic device, comprising:
obtaining motion information from a motion sensor of the ear-wearable electronic device;
generating a first respiration rate estimate using the motion information;
obtaining photoplethysmographic (PPG) data from a PPG sensor of the ear-wearable electronic device;
generating a second respiration rate estimate using the PPG data; and
producing a respiration rate estimate using the first and second respiration rate estimates.

2. The method of claim 1, wherein generating the first respiration rate estimate comprises:
filtering the motion information using a bandpass filter configured to pass frequencies in a frequency range consistent with human breathing; and
applying a sinus fitting to the bandpass-filtered motion information to generate the first respiration rate estimate.

3. The method of claim 1, wherein generating the second respiration rate estimate comprises:
filtering the PPG data using a high pass filter having a specified cutoff frequency;
performing a time domain-to-frequency domain transform on the high pass-filtered PPG data;
performing peak and local minimum detection on the transformed PPG data; and
applying a sinus fitting to heights of peaks of the transformed PPG data to generate the second respiration rate estimate.

4. The method of claim 1, comprising:
capturing the motion information in a plurality of temporally spaced first windows;
capturing the PPG data in a plurality of temporally spaced second windows; and
selecting one of the first windows and one of the second windows for processing based on predefined spectral content criteria;
wherein the predefined spectral content criteria comprises a highest peak in a spectral domain in the range of about 0.1 Hz to about 0.5 Hz.

5. The method of claim 1, comprising:
performing a test of motion sensor signal integrity; and
performing a test of PPG sensor signal integrity.

6. The method of claim 1, wherein producing the respiration rate estimate comprises processing the first and second first respiration rate estimates using a fusion algorithm to produce the respiration rate estimate.

7. The method of claim 1, wherein producing the respiration rate estimate comprises:
comparing the second respiration rate to a threshold;
outputting the second respiration rate estimate in response to the second respiration rate exceeding the threshold; and
outputting the first respiration rate estimate in response to the second respiration rate failing to exceed the threshold.

8. The method of claim 1, comprising:
performing an activity status test using the motion information and PPG data;
determining if a measure of the wearer's physical activity based on the motion information is consistent with the wearer's respiration rate estimated using the PPG data; and
performing a validity test of the second respiration rate estimate by comparing the second respiration rate estimate to a threshold.

9. The method of claim 1, comprising communicating respiration rate data alone or in combination with other physiologic data to one or both of an external electronic device and a cloud database.

10. The method of claim 9, comprising generating one or more of an early warning score, long term analyses, and respiration rate trending reports by one or both of the external electronic device or a cloud processor.

11. An ear-wearable electronic device, comprising:
a motion sensor configured to generate motion information and a first respiration rate estimated using the motion information;
a photoplethysmographic (PPG) sensor configured to generate PPG data and a second respiration rate estimate using the PPG data;
a processor configured to produce a respiration rate estimate using the first and second respiration rate estimates; and
a communication device configured to communicate the respiration rate estimate to one or both of an external electronic device and a cloud database.

12. The device of claim 11, comprising:
bandpass filter configured to pass frequencies of the motion information in a frequency range consistent with human breathing; and
a sinus fitting module configured to apply sinus fitting to the bandpass-filtered motion information to generate the first respiration rate estimate.

13. The device of claim 11, wherein the second respiration rate estimate is generated by use of:
a high pass filter having a specified cutoff frequency configured to filter the PPG data;
a peak and local minimum detector configured to perform peak and local minimum detection on frequency transformed PPG data; and
a sinus fitting module configured to apply a sinus fitting to heights of peaks of the frequency transformed PPG data to generate the second respiration rate estimate.

14. The device of claim 11, comprising:
a memory configured to capture the motion information in a plurality of temporally spaced first windows and to capture the PPG data in a plurality of temporally spaced second windows;
wherein the processor is configured to select one of the first windows and one of the second windows for processing based on predefined spectral content criteria, and the predefined spectral content criteria comprises a highest peak in a spectral domain in the range of about 0.1 Hz to about 0.5 Hz.

15. The device of claim 11, wherein the processor is configured to:
perform a test of motion sensor signal integrity; and
perform a test of PPG sensor signal integrity.

16. The device of claim 11, comprising a data fusion module configured to process the first and second first respiration rate estimates using a fusion algorithm to produce the respiration rate estimate.

17. The device of claim 16, wherein the data fusion module is configured to:
compare the second respiration rate to a threshold;
output the second respiration rate estimate in response to the second respiration rate exceeding the threshold; and
output the first respiration rate estimate in response to the second respiration rate failing to exceed the threshold.

18. The device of claim 11, wherein the processor is configured to:
perform an activity status test using the motion information and PPG data; and
determine if a measure of the wearer's physical activity based on the motion information is consistent with the wearer's respiration rate estimated using the PPG data.

19. The device of claim 11, wherein the processor is configured to perform a validity test of the second respiration rate estimate by comparing the second respiration rate estimate to a threshold.

20. The device of claim 11, wherein the processor is configured to calculate the respiration rate estimate of the wearer in response to successful signal integrity, activity, and validity tests.

21. The device of claim 11, wherein the processor is configured to communicate respiration rate data alone or in combination with other physiologic data to one or both of an external electronic device and a cloud database.

22. The device of claim 21, wherein one or both of the external electronic device and a cloud processor are configured to generate one or more of an early warning score, long term analyses, and respiration rate trending reports.
